(19) United States
(12) Patent Application Publication
(10) Pub. No.: US 2015/0031646 A1
(43) Pub. Date: Jan. 29, 2015

(54) CARRIER ENHANCING ABSORPTION OF MEDICATION

(71) Applicant: MING-CHEN LEE, Taipei City (TW)

(72) Inventor: MING-CHEN LEE, Taipei City (TW)

(21) Appl. No.: 13/950,806

(22) Filed: Jul. 25, 2013

(51) Int. Cl. A61K 47/36; A61K 31/7008

(52) U.S. Cl. CPC A61K 47/36; A61K 31/7008
USPC 514/62; 514/777

(57) ABSTRACT

A carrier enhancing absorption of medication includes a target medication, an emulsifier, a small-molecular hyaluronic acid (HA) component, an absorption enhancer and a cream base, wherein the carrier promotes infiltration of target medication from skin over affected area and enhance the absorption efficacy of human body by synergistic interaction.

### BACKGROUND OF THE INVENTION

1. Field of the Invention

The present invention relates to carriers enhancing absorption of medication, and more particularly, to a carrier that enhances absorption of medication such as glucosamine, non-steroid anti-inflammatory drugs, painkillers or hormone preparations through synergistic interaction of the composition.

2. Description of the Related Art

As a drug delivery system (DDS), ointment is proven effective in delivering active ingredients therein into skin. Thus, many medical products are made into cream or ointment products. With the characteristic of absorbability for skin, after the medical products are applied on the skin of the affected area, active ingredients thereof can be effectively and directly absorbed and utilized. Compared with other drug delivery methods such as oral formulations or injections, directly applying the medication on affected area has greater accuracy and effective proportion for active ingredients to function on the target area. For terminal cancer patients, consuming oral doses or injection is theoretically impracticable; instead, applying on skin is a more appropriate method for medical ingredients to be absorbed. Beside, as for oral formulation of glucosamine, it is proven effective in promoting joint cartilage growth, and thus widely used to cure or alleviate joint degeneration. However, to achieve the efficacy of oral consuming, glucosamine must be in extremely large doses; besides, the active ingredients are difficult to reach target organs. Therefore, applications other than oral formulation are needed.

It is known that cream is made by dispersing aqueous phase and oil phases in the presence of an emulsifier. However, the high stickiness and thickness often make traditional cream disliked by users. In addition, it is believed that small-molecular polyethylene glycol (PEG) is possible to cause cancer. Further, skin is an organ for protecting human body, so that formulations except small-molecular and steroid medication are difficult to be absorbed by skin.

### SUMMARY OF THE INVENTION

For improving such shortcomings, the inventor herein proposes an absorption improving carrier with advanced formulation. To aqueous or oil components added therein, emulsification can reduce particle diameters and increase absorbability. By working with additional absorption enhancers and carriers, skin absorption thereto can be further improved, in order to enhance the absorption of medical ingredients.

The present invention thus provides a carrier enhancing absorption of medication, which comprises:

a target medication;

an emulsifier;

a small-molecular hyaluronic acid;

an absorption enhancing component; and

a cream base;

components of the carrier cooperatively interact to promote the infiltration of the target medication through skin of the affected region, thereby enhancing the absorption efficiency of human body.

The main objective of the present invention is that based on the principle of DDS, the larger the emulsified particle is, the more the active ingredients are absorbed by skin. To enhance the absorption of the target medication, the emulsifier and the cream base of the present invention are characterized by small emulsified particle and easy absorption for human skin. Besides, the target medication with small-molecular hyaluronic acid as the carrier may infiltrate the human skin and be absorbed more easily.

Another objective of the present invention is that an oligogeline and a blood circulation enhancer can be further added for forming a film patch over the applied area for protection, and as well promoting the transdermal absorption of the target medication, blood circulation, metabolism and absorption efficacy of active ingredients.

Still another objective of the present invention is that existing cosmetics or cream composition usually use polyethylene glycol (PEG) as a humectant, but PEG has been considered as a carcinogen by the U.S. FDA in recent years. Therefore, the formulation of the present invention applies PEG free ingredients, thereby lowering user's misgivings toward health and safety while using the products.

### BRIEF DESCRIPTION OF THE DRAWINGS

FIG. 1 is a statistic chart of experiment for derma infiltration of the present invention.

### DETAILED DESCRIPTION OF THE INVENTION

The present invention relates to a carrier enhancing absorption of medication, which comprises:

a) a target medication:

The target medication is selected from a group of medications such as glucosamine HCL, non-steroid anti-inflammatory drugs, painkillers and hormone preparations.

b) an emulsifier:

Most existing cosmetics or cream compositions usually use polyethylene glycol (PEG) as an humectant and thickener; however, small-molecular polyethylene glycol (PEG) is considered as a carcinogen by U.S. FDA in recent years. The formulation of the present invention thus applies PEG free components, such as a natural bionic-smectic liquid crystal emulsifier, which is characterized by the small-sized particle and high absorbability for human skin. In addition, polyethylene glycol (PEG) and ethylene oxide (EO) are also absent therein, so the impact on human body and the environment is thus decreased. For example, an emulsifier of Emulgade® PL68/50 is used in the present invention.

As a principle of DDS, the larger the amount of active ingredients is, the more the active ingredients are absorbed by skin. The emulsifier used in the present invention is bionic-smectic liquid crystal emulsifier from natural resource, wherein the emulsifying efficacy is remarkable. And the particle thus produced is far smaller than the particle of ordinary emulsifier, so the particle is absorbed by human body more easily.

c) a small-molecular hyaluronic acid (HA) component:

The small-molecular hyaluronic acid (HA) has an average molecular weight of 5000-6000, with a particle size of about 25 nanometers, which can readily pass through the intervals between keratinocyte cells as a carrier of medication. Therefore, it is easily absorbed by skin through sweat glands, cell intervals and pores. Small-molecular hyaluronic acid, similar to big-molecular hyaluronic acid, is effective in moisturizing skin and reducing wrinkles, and is further effective in suppressing inflammation and oxidation. The hyaluronic acid (HA) component is present in an amount of 0.1 wt %-1 wt %, based on the total weight of the carrier.

d) an absorption enhancing component:

The absorption enhancing component is herein dimethyl isosorbide (DMI), which is an organic solvent suitable for medication, capable of facilitating uniform dispersion of active ingredients, and thus providing better absorbability. It is present in an amount of 1 wt %-36 wt %, based on the total weight of the carrier.

e) a cream base;

f) an oligogeline component:

Oligogeline is a natural gel extract derived from chondrus crispus seaweed, also known as “marine bandage” for its effectiveness in skin health restoration, and capable of facilitating uniform dispersion of cream base ingredients. When applied on the target area, a film patch is formed after contacting the air from the surface of the carriers, thereby attaching the carrier to the skin of the target area. Further, it prevents the moisture from escaping from the carrier, and keeps the carrier from falling off or lost due to the fricative motions from the user, thereby increasing the absorbability of active ingredients of the target medication. The oligogeline is present in an amount of 1 wt %-10%, based on the total weight of the carrier.

g) a blood circulation promoting component:

The blood circulation promoting component is herein menthol or capsicum frutescens. Menthol and capsicum frutescens promote blood circulation, metabolism, and thereby enhancing skin absorption of active ingredients. Also, it adds fresh smell and cooling sensation to the skin where it is applied, so as to ease pain and uncomfortableness at the affected area. Menthol or capsicum frutescens are present in an amount of 0.1 wt %-1 wt %, based on the total weight of the carrier.

With the formula disclosed above, the present invention is designed for directly applying on the skin over the target area, whereby the active ingredients of the target medication is absorbed by the skin, and directly act on the target tissue for improving target conditions, and enhancing the absorbability of the medication.

For proving that the invention actually has the effects abovementioned, the inventor further proceeded following experiments of absorbability of the target medication such as glucosamine, and entrusted a credible third-party medical organization to process the experimental tests.

1) Organization: Mackay Memorial Hospital, Innovation & Incubation Center

2) Executor: Mackay Memorial Hospital, Department of Medical Research, Technical Officer, Chuang Chih Kuang

3) Experiment Design: Cortex Treatment of Nude Mouse

The research uses cortex from the back of nude mouse as a research target. The cortex is cut off from the body of the animal, and the muscle on the cortex is removed by a surgical knife; afterward, neutral cleanser diluted with salt water is applied to cleanse oil and filth on the cortex. Next, the cortex is washed with normal saline for 3 to 5 times, and cut into pieces with perimeter of 2 cm×2 cm.

4) Transdermal Absorption Experiment:

All samples are tested repeatedly for six times, applying vertical Franz cell to test if the glucosamine is delivered transdermally by the formulation under the room temperature. The vertical Franz cell is separated into an upper chamber and a lower chamber, wherein the upper chamber is a donor chamber and the lower chamber is a receptor chamber. Before clipping in the subject cortex, the receptor chamber is filled with normal saline and settled with a miniature rotor. Next, the cortex is flatly placed and clipped on the donor chamber with a metal clip. The formulation of 2 ml is then added into the donor chamber, and the donor chamber is sealed with a parafilm. Afterward, the Franz cell is placed on an electromagnetic stirrer to spin the miniature rotor. Sample of 50 ul in the receptor chamber is taken out from the sampling port after 0, 2, 4, 6 and 24 hours, respectively.

5) Experimental Groups and Control Groups:

Table 1 is the formulation list of a first embodiment of the present invention, wherein group D is control group.

TABLE 1
Formulation List of the Embodiment

| | | D | E | F |
|---|---|---|---|---|
| Trade Name | INCI Name | % | % | % |
| DMI | Dimethyl | 1-15 | 1-15 | 15-36 |
| | isosorbide | | | |
| Cyclic | Cyclic | 6 | 6 | 6 |
| methicone | methicone | | | |
| 040 | | | | |
| Mineral oil | Mineral oil | 3-6 | 3-6 | 3-6 |
| Dimethicone | Dimethicone | 3 | 3 | 3 |
| 350 | | | | |
| Oligogeline | Chondruscrispus | 1-10 | 1-10 | 1-10 |
| Emulgade PL | Cetearyglucoside | 1-3 | 1-3 | 1-3 |
| 68/50 | (and)cetearyl | | | |
| | alcohol | | | |
| Cetyl alcohol | Cetyl alcohol | 1-2 | 1-2 | 1-2 |
| Cyclic | Cyclic | 2 | 2 | 2 |
| methicone | methicone | | | |
| 1000 | | | | |
| Glycerol | Glycerol | 2 | 2 | 2 |
| Hyaluronate | Hyaluronate | 1-5 | 1-5 | 1-5 |
| acid (10%) | acid | | | |
| Stearic acid | Stearic acid | 1-3 | 1-3 | 1-3 |
| Aloe extract | Aloe barbadensis | 1 | 1 | 1 |
| (10x) | leaf extract | | | |
| Menthanol | Menthanol | 0.1-1 | 0.1-1 | 0.1-1 |
| Xanthan gum | Xanthan gum | 0.1-1 | 0.1-1 | 0.1-1 |
| Ultragel 300 | Polyguaternium-37 | 0.1-1 | 0.1-1 | 0.1-1 |
| Grapefruit | Citrus grandis | 0.3 | 0.3 | 0.3 |
| seed extract | (grapefruit) seed | | | |
| | extract, glycerin | | | |
| α-bisabolol | α-bisabolol | 0.5 | 0.5 | 0.5 |
| Oleoresin | Capsicum | 0.1-0.5 | 0.1-0.5 | 0.1-0.5 |
| capsicum | frutescens | | | |
| Allantoin | Allantoin | 0.3 | 0.3 | 0.3 |
| Eumulgin SG | Sodium stearoyl | 0.1-0.5 | 0.1-0.5 | 0.1-0.5 |
| | glutamate | | | |
| α-tocopherol | α-tocopherol | 0.1 | 0.1 | 0.1 |
| acetate | acetate | | | |
| H2O | Water | Added to | Added to | Added to |
| | | 100% | 100% | 100% |

6) Sample Analysis:

The sample of 50 ul taken out from the receptor chamber of the Franz cell is centrifugated with 12000 rpm for 30 minutes, and the supernatant liquid therein taken out is put into the analyzer tube of Liquid Chromatograph Tandem Mass Spectrometer. The Liquid Chromatograph Tandem Mass Spectrometer is operated under following circumstances: Separation column is in the format of Luna silica 100 A (2×50 mm. 5 um); mobile phase A is 0.01% formic acid, while Mobile phase B is 1 mM NH4OAc+0.1% formic acid in 100% acetonitrile; elution of gradient is presented as Table 3. The main signal appears at about 2 to 3 minutes after elution begins. Measure of area under the signal is calculated with integral and compared with standard curve line to get a value D (ng/ml). By a formula of “D (ng/ml)×volume of receptor chamber V (ml)÷total measure of area of transdermal cortex F (cm²)”, the infiltration ratio P (ng/cm²) is calculated.

TABLE 2
Operational Circumstances of Liquid
Chromatograph Tandem Mass Spectrometer

| | |
|---|---|
| Column: | Luna silica 100 A (2 × 50 mm, 5 um) |
| Mobile phase A: | 0.01% Formic acid |
| Mobile phase B: | 1 mM NH4OAc + 0.1% FA in 100% Acetonitrile |

| Step | Total Time (min) | Flow Rate (μl/min) | A (%) | B (%) |
|---|---|---|---|---|
| 0 | 0.00 | 600 | 10.0 | 90.0 |
| 1 | 0.10 | 600 | 10.0 | 90.0 |
| 2 | 0.50 | 600 | 50.0 | 50.0 |
| 3 | 2.00 | 600 | 50.0 | 50.0 |
| 4 | 2.30 | 600 | 10.0 | 90.0 |
| 5 | 5.00 | 600 | 10.0 | 90.0 |

7) Result:

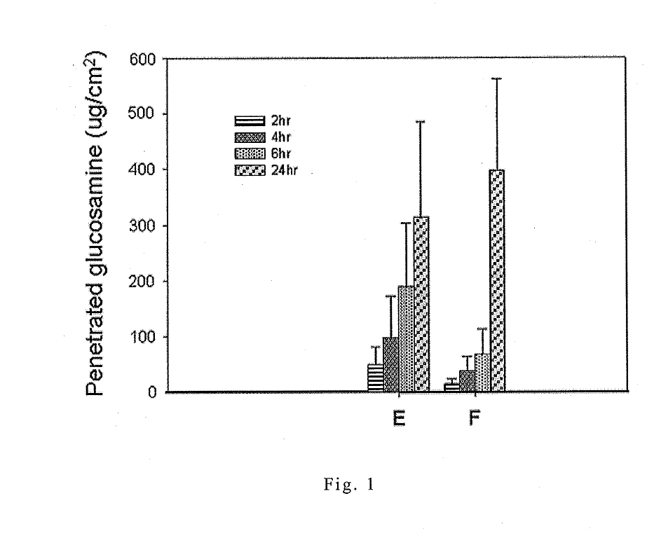

Based on the analysis, sample D does not contain glucosamine (Table 3), Based on the trend of 6-hour-infiltration (FIG. 1), the best transdermal effect occurs with E at 180 ug/cm², while the effect of F is similar at about 50 to 60 ul/cm². In the value of 24-hour-infiltration (Table 1), results of E and F are above 300 ug/cm².

TABLE 3
The statistics of transdermal absorption of formulations E and F,
wherein glucosamine is not contained in D, and thus D is not listed.
(AVE: average value(ng/cm²); SE: standard errors)

| | 1 | 2 | 3 | 4 | 5 | 6 | AVE | SE |
|---|---|---|---|---|---|---|---|---|
| E-2 h | 205732 | 0 | 57325 | 24994 | 287 | 430 | 48128 | 32832 |
| E-4 h | 471631 | 0 | 54471 | 49176 | 576 | 578 | 96072 | 75816 |
| E-6 h | 733210 | 91083 | 237488 | 65598 | 439 | 298 | 188019 | 114672 |
| E-24 h | 339962 | 1071885 | 461118 | 2114 | 444 | 444 | 312661 | 172299 |
| F-2 h | 60764 | 0 | 0 | 16338 | 287 | 287 | 12946 | 9923 |
| F-4 h | 163580 | 0 | 0 | 51774 | 863 | 433 | 36108 | 26843 |
| F-6 h | 280688 | 37261 | 0 | 83303 | 586 | 295 | 67022 | 44782 |
| F-24 h | 375096 | 282274 | 1186624 | 232118 | 25471 | 276317 | 396317 | 165018 |

According to the results above, the cream composition of the present invention actually enhances the absorption of glucosamine after 2, 4, 6 and even 24 hours, thereby achieving the objective efficiently.

What is claimed is:

1. A carrier enhancing absorption of medication, which comprises:
at least one target medication;
an emulsifier;
a small-molecular hyaluronic acid (HA);
an absorption enhancing component; and
a cream base;
the components of the carrier act synergistically to help the active ingredients of the target medication to infiltrate into skin of affected areas, thereby enhancing the absorption of human body.

2. The carrier of claim 1, wherein the target medication is selected from at least one of glucosamine HCL, non-steroid anti-inflammatory drugs, painkillers and hormone preparations.

3. The carrier of claim 1, wherein the emulsifier is a bionic-smectic crystal liquid emulsifier from natural resource.

4. The cream composition of claim 1, wherein the small-molecular hyaluronic acid (HA) component has a molecular weight of 5000-6000, and is present in an amount of 0.1 wt % to 1 wt %, based on the total weight of the carrier.

5. The carrier claim 1, wherein the cream base further comprises an Oligogeline component that is present in an amount of 1 wt % to 10 wt %, based on the total weight of the carrier.

6. The carrier of claim 1, wherein the absorption enhancing component is present in an amount of 1 wt % to 36 wt %, based on the total weight of the carrier.

7. The carrier of claim 6, wherein the absorption enhancing component is dimethyl isosorbide (DMI).

8. The carrier of claim 1, wherein the cream base further comprises a blood circulation promoting component that is present in an amount of 0.1 wt % to 1 wt %, based on the total weight of the carrier.

9. The carrier of claim 8, wherein the blood circulation promoting component is menthol or capsicum frutescens.
